(19) United States
(12) Patent Application Publication
(10) Pub. No.: US 2025/0012936 A1
(43) Pub. Date: Jan. 9, 2025

(54) RADIATION IMAGE CAPTURE APPARATUS, RADIATION IMAGE SHOOTING SYSTEM, AND CONTROL METHOD

(71) Applicant: CANON KABUSHIKI KAISHA, Tokyo (JP)

(72) Inventors: HIDEAKI MORITA, Tokyo (JP); RYOSUKE MIURA, Chiba (JP)

(21) Appl. No.: 18/893,623

(22) Filed: Sep. 23, 2024

Related U.S. Application Data
(63) Continuation of application No. PCT/JP2023/007957, filed on Mar. 3, 2023.

(30) Foreign Application Priority Data
Mar. 28, 2022 (JP) 2022-051870

(51) Int. Cl. G01T 1/29; G01T 1/02; G01T 1/17; G01T 1/24; G01T 7/00

(52) U.S. Cl. CPC G01T 1/2985; G01T 1/026; G01T 1/17; G01T 1/247; G01T 1/2992; G01T 7/005

(57) ABSTRACT

A radiation image capture apparatus including a plurality of pixels which include a detection pixel configured to detect an irradiation amount and an exposure decision unit configured to decide an amount of radiation irradiated from a radiation generation apparatus. The exposure decision unit decides the amount of irradiated radiation based on an output of the detection pixel during radiation irradiation and a correction value that is an output of the detection pixel while the radiation irradiation is not performed. The exposure decision unit obtains the correction value after a read operation of a signal based on radiation during image capture is performed, and in the subsequent image capture, the amount of irradiated radiation is decided based on the output of the detection pixel during the radiation irradiation and the correction value.

### CROSS-REFERENCE TO RELATED APPLICATIONS

This application is a Continuation of International Patent Application No. PCT/JP2023/007957, filed Mar. 3, 2023, which claims the benefit of Japanese Patent Application No. 2022-051870, filed Mar. 28, 2022, both of which are hereby incorporated by reference herein in their entirety.

### TECHNICAL FIELD

The present disclosure relates to a radiation image capture apparatus, a radiation image shooting system, and a control method.

### BACKGROUND ART

A radiation image shooting apparatus having an automatic exposure control (AEC) function has been proposed.

Such a radiation image capture apparatus can measure a radiation dose during irradiation and end the radiation irradiation according to a result of the measurement.

For example, the radiation image capture apparatus monitors a radiation dose by causing only pixels set for radiation detection to operate at a high speed during radiation irradiation. In addition, until a request for starting radiation irradiation is received, to reset dark charges accumulated in each of pixels, the radiation image capture apparatus performs a reset operation for causing each of the pixels to operate in sequence.

PTL 1 discloses a radiation image capture apparatus configured to obtain, during a reset operation of each of pixels before a request for starting radiation irradiation is received, an offset component used for monitoring a radiation dose during the radiation irradiation.

### CITATION LIST

### Patent Literature

PTL 1 Japanese Patent Laid-Open No. 2020-89714

Since the radiation image capture apparatus of PTL 1 is not capable of performing radiation irradiation while an offset component is obtained, depending on timing for a user to instruct shooting, timing for starting shooting and timing for ending the shooting may be delayed.

### SUMMARY OF DISCLOSURE

The present disclosure has been made in view of such an issue in related art, and aims at providing a technique for accurately obtaining an offset component used for monitoring a radiation dose during radiation irradiation while delays in timing for starting shooting and timing for ending the shooting are reduced.

The above-described issue is addressed by a radiation image capture apparatus including a plurality of pixels including a pixel configured to output a signal based on irradiated radiation and an exposure decision unit configured to decide an amount of the radiation irradiated to the radiation image capture apparatus by a radiation generation apparatus, in which the exposure decision unit decides the amount of the radiation irradiated to the radiation image capture apparatus by using a first correction value obtained based on a value of a signal read from the pixel while the radiation from the radiation generation apparatus is irradiated to the radiation image capture apparatus and a signal read from the pixel while the radiation from the radiation generation apparatus is not irradiated to the radiation image capture apparatus, and the exposure decision unit obtains the first correction value after a read operation of a signal based on the radiation irradiated from the radiation generation apparatus in first image shooting for shooting an image based on the radiation is performed and decides, by using the value of the signal read from the pixel and the first correction value, the amount of the radiation irradiated to the radiation image capture apparatus when second image shooting, which is to be performed after the first image shooting, for obtaining the image based on the radiation is performed.

Further features of the present disclosure will become apparent from the following description of exemplary embodiments with reference to the attached drawings.

### BRIEF DESCRIPTION OF DRAWINGS

FIG. 1 illustrates a configuration of a radiation image capture apparatus according to a first embodiment.

FIG. 2 illustrates a configuration of an amplification unit according to the first embodiment.

FIG. 3 is a plan view illustrating a configuration of each of pixels according to the first embodiment.

FIG. 4A is a cross sectional view illustrating the configuration of each of the pixels according to the first embodiment.

FIG. 4B is a cross sectional view illustrating the configuration of each of the pixels according to the first embodiment.

FIG. 5 illustrates a configuration example of a radiation image capture system including a radiation image capture apparatus according to the first embodiment.

FIG. 6 illustrates an operation of the radiation image capture apparatus according to the first embodiment.

FIG. 7 is an explanatory diagram for describing an operation of obtaining a correction value according to the first embodiment.

FIG. 8 is an explanatory diagram for describing the operation of obtaining the correction value according to the first embodiment.

FIG. 9 is an explanatory diagram for describing the operation of obtaining the correction value according to the first embodiment.

FIG. 10 is a flow diagram for describing the operation of obtaining the correction value according to the first embodiment.

FIG. 11 is a flow diagram illustrating an operation of the radiation image capture apparatus according to a second embodiment.

FIG. 12 is a flow diagram illustrating the operation of the radiation image capture apparatus according to a third embodiment.

FIG. 13 illustrates a configuration example of a radiation image shooting system according to a fourth embodiment.

### DESCRIPTION OF EMBODIMENTS

Embodiments of the present disclosure will be described below with reference to the accompanying drawings. Similar elements through various embodiments are allocated with the same reference signs, and duplicated descriptions will not be repeated. In addition, respective embodiments can be appropriately modified and combined with each other.

### First Embodiment

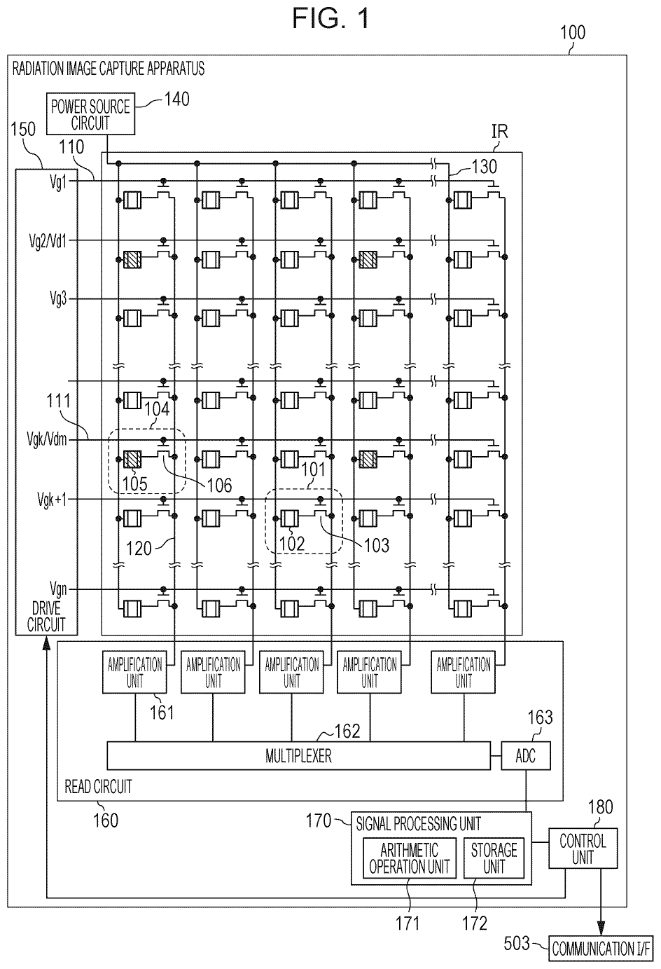

FIG. 1 illustrates a configuration example of a radiation image capture apparatus 100 according to a first embodiment of the present disclosure. The radiation image capture apparatus 100 has a plurality of pixels set in array in an image capture region IR to configure a plurality of rows and a plurality of columns, a plurality of drive lines 110, and a plurality of signal lines 120. The plurality of drive lines 110 are arranged so as to correspond to the plurality of rows of the pixels, and each of the drive lines 110 corresponds to any one of the pixel rows. The plurality of signal lines 120 are arranged so as to correspond to the plurality of columns of the pixels, and each of the signal lines 120 corresponds to any one of the pixel columns.

The plurality of pixels include a plurality of image capture pixels 101 used to obtain a radiation image, one or more detection pixels 104 used to monitor an irradiation amount of radiation, and one or more correction pixels 107 used to correct the irradiation amount of the radiation. A sensitivity of the correction pixel 107 to the radiation is lower than a sensitivity of the detection pixel 104 to the radiation.

The image capture pixel 101 includes a conversion element 102 configured to convert radiation into an electric signal and a switch element 103 which connects the corresponding signal line 120 and the conversion element 102 to each other. The detection pixel 104 includes a conversion element 105 configured to convert radiation into an electric signal and a switch element 106 which connects the corresponding signal line 120 and the conversion element 105 to each other.

The detection pixels 104 are arranged to be included in the rows and the columns which are configured by the plurality of image capture pixels 101. The correction pixel 107 includes a conversion element 108 configured to convert radiation into an electric signal and a switch element 109 which connects the signal line 120 and the conversion element 108 to each other. The correction pixels 107 are arranged to be included in the rows and the columns which are configured by the plurality of image capture pixels 101.

In FIG. 1 and the subsequent drawings, the image capture pixel 101, the detection pixel 104, and the correction pixel 107 are distinguished from one another by applying mutually different hatchings to the conversion element 102, the conversion element 105, and the conversion element 108.

The conversion element 102, the conversion element 105, and the conversion element 108 may be constituted by a scintillator configured to convert radiation into light and a photoelectric conversion element configured to convert light into an electric signal. In general, the scintillator is formed into a sheet-like shape so as to cover the image capture region IR and shared by the plurality of pixels. Instead of this, the conversion element 102, the conversion element 105, and the conversion element 108 may be constituted by a conversion element configured to directly convert radiation into an electric signal.

The switch element 103, the switch element 106, and the switch element 109 may include, for example, a thin film transistor (TFT) in which an active region is made of a semiconductor such as amorphous silicon or polycrystalline silicon.

A first electrode of the conversion element 102 is connected to a first main electrode of the switch element 103, and a second electrode of the conversion element 102 is connected to a bias line 130. One bias line 130 extends in a column direction and is commonly connected to second electrodes of the plurality of conversion elements 102 set in array in the column direction. The bias line 130 receives a bias voltage Vs from a power source circuit 140. A second main electrode of the switch element 103 of one or more image capture pixels 101 included in one column is connected to one signal line 120. A control electrode of the switch element 103 of one or more image capture pixels 101 included in one row is connected to one the drive line 110.

The detection pixel 104 and the correction pixel 107 also have a pixel configuration similar to that of the image capture pixel 101 and are connected to the corresponding drive line 110 and the corresponding signal line 120. The detection pixel 104 and the correction pixel 107 are exclusively connected to the signal line 120. That is, the correction pixel 107 is not connected to the signal line 120 to which the detection pixel 104 is connected. On the other hand, the detection pixel 104 is not connected to the signal line 120 to which the correction pixel 107 is connected. The image capture pixel 101 may be connected to the same signal line 120 as the detection pixel 104 or the correction pixel 107.

A drive circuit 150 is configured to supply a drive signal to a pixel set as a drive target through the plurality of drive lines 110 following a control signal from a control circuit 180. According to the present embodiment, the drive signal is a signal for turning on the switch element included in the pixel set as the drive target.

The switch element in each of the pixels turns on by a signal at a high level and turns off by a signal at a low level. For this reason, this signal at the high level is called a drive signal. Since the drive signal is supplied to the pixel, a state is established in which the signal accumulated in the conversion element of this pixel can be read by a read circuit 160. In a case where the drive line 110 is connected to at least one of the detection pixel 104 and the correction pixel 107, the above drive line 110 is called a detection drive line 111.

The read circuit 160 is configured to read signals from the plurality of pixels through the plurality of signal lines 120. The read circuit 160 includes a plurality of amplification units 161, a multiplexer 162, and an analog-to-digital converter (hereinafter, an AD converter) 163. Each of the plurality of signal lines 120 is connected to the corresponding amplification unit 161 among the plurality of amplification units 161 of the read circuit 160. One signal line 120 corresponds to one amplification unit 161.

The multiplexer 162 selects the plurality of amplification units 161 in a predetermined order and supplies a signal from the selected amplification unit 161 to the AD conversion unit 163. The AD conversion unit 163 converts the supplied signal into a digital signal to be output.

The signal read from the image capture pixel 101 is supplied to the signal processing unit 170 and subjected to processing such as an arithmetic operation or storage by the signal processing unit 170. Specifically, the signal processing unit 170 includes an arithmetic operation unit 171 and a storage unit 172. The arithmetic operation unit 171 generates a radiation image based on the signal read from the image capture pixel 101 and supplies the radiation image to the control circuit 180.

The signals read from the detection pixel 104 and the correction pixel 107 are supplied to the signal processing unit 170 and subjected to processing such as an arithmetic operation or storage by the above arithmetic operation unit 171. Specifically, the signal processing unit 170 outputs information indicating radiation irradiation to the radiation image capture apparatus 100 based on the signals read from the detection pixel 104 and the correction pixel 107. For example, the signal processing unit 170 detects the radiation irradiation to the radiation image capture apparatus 100 and decides an irradiation amount of the radiation and/or an accumulated irradiation amount.

The control circuit 180 controls the drive circuit 150 and the read circuit 160 based on the information from the signal processing unit 170. The control circuit 180 controls, for example, start and end of exposure (accumulation of charges corresponding to irradiated radiation by the image capture pixel 101) based on the information from the signal processing unit 170.

To decide the irradiation amount of the radiation, the control circuit 180 controls the drive circuit 150 to scan only the detection drive line 111 to establish a state in which only the signals from the detection pixel 104 and the correction pixel 107 can be read. Next, by controlling the read circuit 160, the control circuit 180 reads the signals in the column corresponding to the detection pixel 104 and the correction pixel 107 to be output as information indicating the irradiation amount of the radiation. With such an operation, the radiation image capture apparatus 100 can obtain irradiation information in the detection pixel 104 during the radiation irradiation.

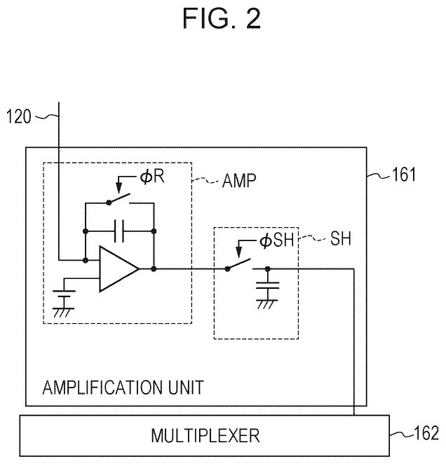

FIG. 2 illustrates a detailed circuit configuration example of the amplification unit 161. The amplification unit 161 includes a differential amplifier circuit AMP and a sample and hold circuit SH. The differential amplifier circuit AMP amplifies a signal appearing on the signal line 120 and outputs the signal. The control circuit 180 can reset a potential of the signal line 120 by supplying a control signal φR to a switch element of the differential amplifier circuit AMP.

An output from the differential amplifier circuit AMP can be held by the sample and hold circuit SH. The control circuit 180 causes the sample and hold circuit SH to hold the signal by supplying a control signal φSH to a switch element of the sample and hold circuit SH. The signal held by the sample and hold circuit SH is to be read by the multiplexer 162.

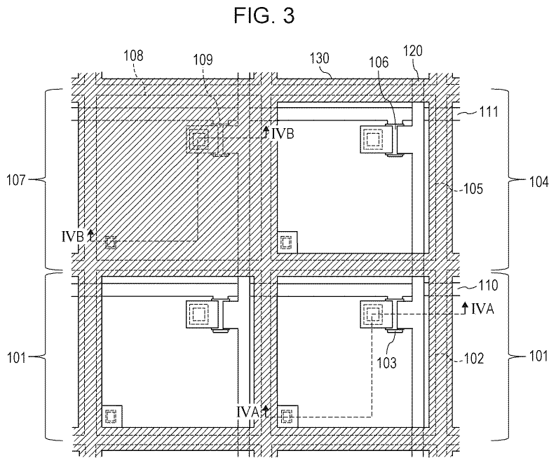

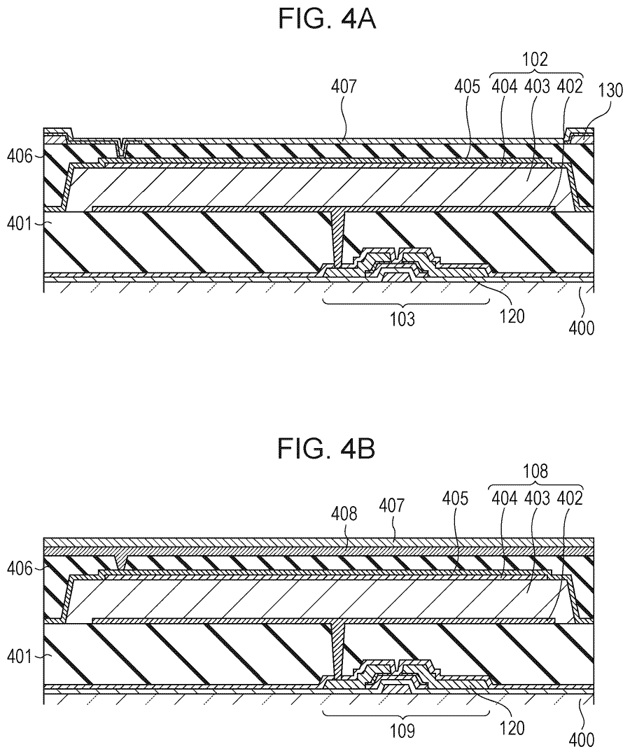

A structural example of the pixels of the radiation image capture apparatus 100 will be described with reference to FIG. 3, FIG. 4A, and FIG. 4B. FIG. 3 is a plan view illustrating a configuration of the image capture pixel 101, the detection pixel 104, and the correction pixel 107 in the radiation image capture apparatus 100. The plan view is equivalent to orthographic projection onto a plane parallel to the image capture region IR of the radiation image capture apparatus 100. As indicated by hatching, a metallic layer is arranged on the conversion element 108 of the correction pixel 107, and the conversion element 108 is shielded from light by this metallic layer.

FIG. 4A is a cross sectional view illustrating the image capture pixel 101 along an IVA-IVA line of FIG. 3. The cross sectional view of the detection pixel 104 is similar to the cross sectional view of the image capture pixel 101. The switch element 103 is arranged on an insulating support substrate 400 such as a glass substrate. The switch element 103 may be a thin film transistor (TFT). An interlayer insulating layer 401 is arranged on the switch element 103. The conversion element 102 is arranged on the interlayer insulating layer 401. This conversion element 102 is a photoelectric conversion element capable of converting light into an electric signal.

The conversion element 102 is constituted by an electrode 402, a P-type intrinsic N-type (PIN) photodiode 403, and an electrode 404, for example. The conversion element 102 may be constituted by a metal insulator semiconductor (MIS) type sensor instead of a PIN type photodiode.

A protective film 405, an interlayer insulating layer 406, the bias line 130, and a protective film 407 are arranged in the stated order on the conversion element 102. A flattening film and the scintillator which are not illustrated in the drawing are arranged on the protective film 407. The photodiode 404 is connected to the bias line 130 via a contact hole. As a material of the electrode 404, indium tin oxide (ITO) having optical transmittance is used, and the light converted from the radiation by the scintillator which is not illustrated in the drawing can pass therethrough.

FIG. 4B is a cross sectional view illustrating the correction pixel 107 along an IVB-IVB line of FIG. 3. The correction pixel 107 may be different from the image capture pixel 101 and the detection pixel 104 in that the conversion element 108 is covered by a light shielding member 408, and other aspects may be the same.

The light shielding member 408 is formed by the same metallic layer as the bias line 130, for example. Since the conversion element 108 of the correction pixel 107 is covered by the light shielding member 408, a sensitivity of the correction pixel 107 to radiation is substantially lower than a sensitivity of the image capture pixel 101 and a sensitivity of the detection pixel 104 to radiation. It can also be mentioned that the charges accumulated in the conversion element 108 of the correction pixel 107 are not caused by radiation.

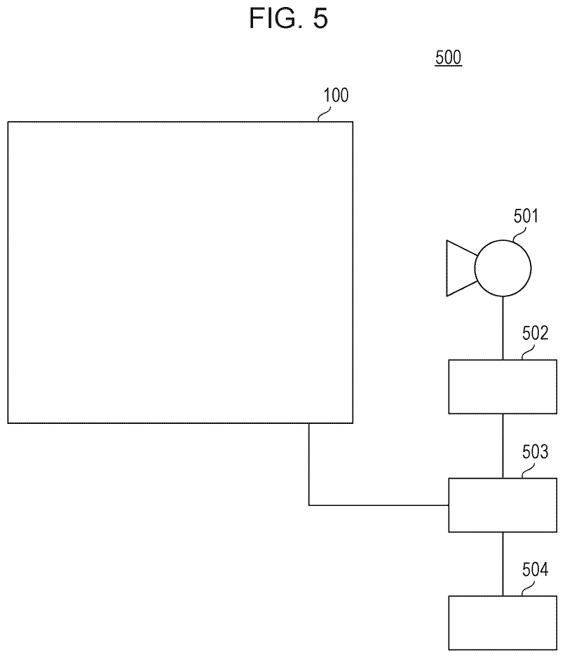

FIG. 5 illustrates a configuration example of a radiation image capture system 500 including the radiation image capture apparatus 100. The radiation image capture system 500 includes the radiation image capture apparatus 100, a radiation source 501, a radiation source interface 502, a communication interface 503, and a controller 504.

A dose, an upper limit of an irradiation time period (ms), a tube current (mA), a tube voltage (kV), a region of interest (ROI) that is a region where radiation is to be monitored, and the like are input to the controller 504. When an exposure switch attached to the radiation source 501 is operated, the controller 504 transmits a start request signal to the radiation image capture apparatus 100. The start request signal is a signal for requesting irradiation start of radiation.

In response to reception of the start request signal, the radiation image capture apparatus 100 starts to prepare acceptance of radiation irradiation. Once the preparation is ready, the radiation image capture apparatus 100 transmits a start possible signal to the radiation source interface 502 via the communication interface 503. The start possible signal is a signal for notification indicating that it is possible to start the radiation irradiation. In response to reception of the start possible signal, the radiation source interface 502 causes the radiation source 501 to start the radiation irradiation.

Herein, before the exposure switch attached to the radiation source 501 is operated, the controller 504 may transmit the start request signal to the radiation image capture apparatus 100. In response to reception of the start request signal, the radiation image capture apparatus 100 starts to prepare acceptance of the radiation irradiation. When the exposure switch attached to the radiation source 501 is operated, the radiation source interface 502 transmits the start request signal to the radiation image capture apparatus 100.

The radiation image capture apparatus 100 transmits information indicating that shooting has been started to the controller 504 via the communication interface 503. Herein, the information may be a signal or may be abstracted information such as a packet. Furthermore, once the preparation is ready, the radiation image capture apparatus 100 transmits the start possible signal to the radiation source interface 502 via the communication interface 503.

At this time, the radiation image capture apparatus 100 may transmit a previously decided preparation time period, and the radiation source interface 502 may wait for the preparation time period since the start possible signal has been received and then transmit an irradiation start signal to the radiation source 501. With such a configuration, advantages are attained that a communication amount immediately after the exposure switch can be lowered, and an exposure delay which will be described below may be reduced.

When a threshold of an integrated value of doses of the irradiated radiation has reached, the radiation image capture apparatus 100 transmits an end request signal to the radiation source interface 502 via the communication interface 503. The end request signal is a signal for requesting end of the radiation irradiation. In response to reception of the end request signal, the radiation source interface 502 causes the radiation source 501 to end the radiation irradiation.

A threshold of the dose is decided by the control circuit 180 based on an input value of the dose, a radiation irradiation intensity, a communication delay between each unit, a processing delay, and the like. When the radiation irradiation time period has reached the input upper limit of the radiation time period, the radiation source 501 stops the radiation irradiation even when the end request signal is not received.

After the radiation irradiation is stopped, the radiation image capture apparatus 100 sequentially scans the drive lines 110 to which only the image capture pixel 101 is connected (the drive lines 110 other than the detection drive lines 111), and an image signal of each of the image capture pixels 101 is read by the read circuit 160 to obtain a radiation image.

The charges accumulated in the detection pixel 104 have been read during the radiation irradiation, and the correction pixel 107 is shielded from light, so that the signals from these pixels are unavailable for the formation of the radiation image. In view of the above, the signal processing unit 170 of the radiation image capture apparatus 100 interpolates pixel values at positions of these pixels by performing interpolation processing using the pixel values of the image capture pixels 101 in the surrounding of the detection pixel 104 and the correction pixel 107.

Herein, the radiation image capture apparatus 100 may perform shooting by way of synchronous shooting and asynchronous shooting. The synchronous shooting is shooting in which shooting timing is coordinated by exchanging electric synchronous signals or the like with the radiation source 501 via the radiation image capture apparatus 100 and the radiation source interface 502. The asynchronous shooting is shooting in which the radiation image capture apparatus 100 senses radiation incidence and starts shooting without exchanging electric synchronous signals or the like with the radiation image capture apparatus 100 and the radiation source 501.

In the asynchronous shooting, without the provision of the radiation source interface 502, when radiation 600 is irradiated from the radiation source 501, the radiation image capture apparatus 100 automatically accumulates image signals (charges) to generate a radiation image. According to the present embodiment, this radiation image shooting system in an automatic detection mode without the provision of the radiation source interface 502 is also applicable. The radiation image shooting apparatus may transfer the radiation image for each shooting in the asynchronous shooting or may save the shot image in the storage unit 172 without transferring the shot image for each shooting.

The radiation image shooting apparatus 100 may have a plurality of shooting modes. For example, a shooting mode of the radiation image capture apparatus 100 in the synchronous shooting may be set as a first shooting mode. In addition, for example, a shooting mode in which the radiation image is transferred to the controller 504 for each shooting in the asynchronous shooting may be set as a second shooting mode. Furthermore, a shooting mode in which the radiation image is not transferred for each shooting in the asynchronous shooting may be set as a third shooting mode.

In other words, the first shooting mode is a shooting mode in which the shooting is performed while the controller 504 causes the radiation image capture apparatus 100 and the radiation source 501 to operate in a collaboration manner in the configuration of the radiation image capture apparatus 100.

The second shooting mode is a shooting mode in which the radiation source interface 502 is not provided in the configuration of the radiation image capture apparatus 100, and when the radiation 600 is irradiated from the radiation source 501, the radiation image capture apparatus 100 automatically accumulates image signals (charges) to generate a radiation image.

In the third shooting mode, shooting can be performed in the configuration without the provision of the controller 504 and the communication interface 503 as well as the radiation source interface 502. In this case, the radiation image capture apparatus 100 and the controller 504 do not necessarily need to communicate information such as an image and a shooting protocol for each shooting, and a radiation generation apparatus alone can obtain the radiation image. In addition, in the third shooting mode, the radiation image capture apparatus 100 can efficiently perform shooting by storing the shot radiation image in the storage unit 172 without communicating with the controller 504 for each shooting.

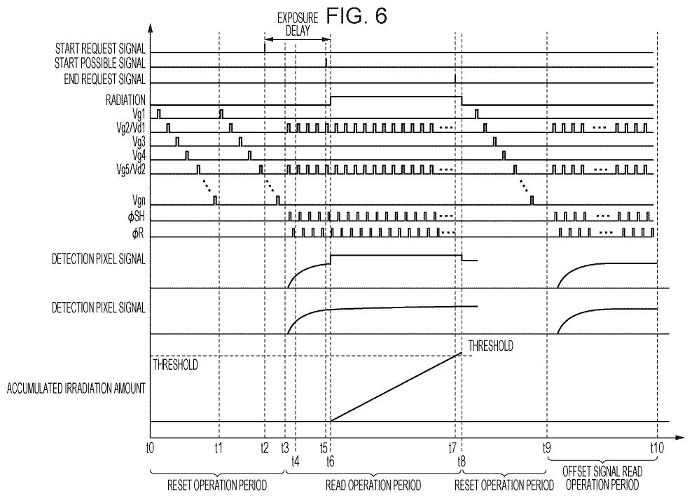

An operation example of the radiation image capture apparatus 100 will be described with reference to FIG. 6. This operation is executed when the control circuit 180 which controls the drive circuit 150 and the read circuit 160 and the signal processing unit 170 cooperate with each other. In addition, an amount of the radiation irradiated to the radiation image capture apparatus 100 is decided through this operation. For this reason, a combination of the signal processing unit 170 and the control circuit 180 may be called an exposure decision unit.

In FIG. 6, “radiation” indicates whether or not radiation is irradiated to the radiation image capture apparatus 100. In the case of low, the radiation is not irradiated, and in the case of high, the radiation is irradiated.

Herein, “Vg1” to “Vgn” indicate drive signals to be supplied from the drive circuit 150 to the plurality of drive lines 110, and “Vgk” corresponds to the drive line 110 in the k-th row (k=1, . . . , a total number of drive lines). As described above, part of the plurality of drive lines 110 is also called the detection drive line 111. The j-th detection drive line 111 is represented as “Vdj” (j=1, . . . , a total number of detection drive lines).

Herein, φSH denotes a level of the control signal supplied to the sample and hold circuit SH of the amplification unit 161. In addition, φR denotes a level of the control signal supplied to the differential amplifier circuit AMP of the amplification unit 161. A “detection pixel signal” indicates a value of a signal read from the detection pixel 104. A “correction pixel signal” indicates a value of a signal read from the correction pixel 107. An “accumulated irradiation amount” indicates an integrated value of the radiation irradiated to the radiation image capture apparatus 100. A method of deciding this integrated value will be described below.

At time t0, the control circuit 180 starts a reset operation of the plurality of pixels. The reset operation refers to an operation of removing charges accumulated in the conversion element of each of the pixels. Specifically, the reset operation refers to an operation of putting the switch element in each of the pixels into a conductive state by supplying the drive signal to the drive line 110. The control circuit 180 resets each of the pixels connected to the drive line 110 in the first row by controlling the drive circuit 150.

Subsequently, the control circuit 180 resets each of the pixels connected to the drive line 110 in the second row. The control circuit 180 repeats this operation up to the drive line 110 in the last row. At time t1, after the reset operation of the drive line 110 in the last row is ended, the control circuit 180 repeats the reset operation again from the drive line 110 in the first row.

At time t2, the control circuit 180 receives the start request signal from the controller 504. In response to the reception of the start request signal, the control circuit 180 performs the reset operation up to the last row and ends the reset operation. Before the reset operation is performed up to the last row, the control circuit 180 may end the reset operation and shift to the next processing.

For example, in a case where the start request signal is received during the reset operation of the drive line 110 in the k-th row, the control circuit 180 may shift to the next processing without performing the reset operation of the drive lines 110 in the (k+1)-th row and the subsequent rows. In this case, unevenness which may be generated in the radiation image may be reduced by performing adjustment on driving for obtaining a radiation image and image processing on the radiation image.

At time t3, the control circuit 180 starts a decision operation for deciding an amount of radiation during irradiation to the radiation image capture apparatus 100. In the decision operation, the control circuit 180 repeatedly executes a read operation for readout from the detection pixel 104 and the correction pixel 107. Among the read operation performed multiple times, the read operation performed at least once in the first half is performed to decide the correction value, and the read operation repeated in the second half is performed to continuously decide the amount of the radiation at each point in time.

The read operation is executed on the detection drive line 111 and is not executed on the drive line 110 excluding the detection drive line 111. Specifically, the drive circuit 150 supplies the drive signal to the drive line 110 connected to at least one of the detection pixel 104 and the correction pixel 107 (that is, the detection drive line 111) among the plurality of drive lines 110.

However, the drive circuit 150 does not supply the drive signal to the drive line 110 which is not connected to any of the detection pixel 104 and the correction pixel 107 among the plurality of drive lines 110. In addition, the drive circuit 150 supplies the drive signal to the drive lines 110 connected to at least one of the detection pixel 104 and the correction pixel 107 among the plurality of drive lines 110 at the same time. With this configuration, signals from the plurality of pixels connected to the same signal line 120 are combined and read to the read circuit 160. Since the detection pixel 104 and the correction pixel 107 are exclusively connected to the signal line 120, the read circuit 160 can separately read the signals of the pixels with different sensitivities.

In the single read operation, the control circuit 180 performs the operation during time t3 to time t4. Specifically, the control circuit 180 temporarily supplies the drive signal to the one or more detection drive lines 111. Thereafter, the control circuit 180 holds the signal read from the pixel to the read circuit 160 through the signal line 120 in the sample and hold circuit SH by temporarily setting the control signal φSH to the high level.

Thereafter, the control circuit 180 resets the read circuit 160 (specifically, the differential amplifier circuit AMP of the above amplification unit 161) by temporarily setting the control signal φR to the high level. In a case where a region of interest is set in the image capture region IR, signals from the detection pixels 104 which are not included in this region of interest do not necessarily need to be read.

To decide the correction value, the control circuit 180 performs the read operation a predetermined number of times that is at least once. The signal processing unit 170 decides a correction value Od based on the signal read from the detection pixel 104 by the read operation performed the predetermined number of times and a correction value Oc based on the signal read from the correction pixel 107 by the read operation performed this predetermined number of times.

The decision on the correction value Od will be described in detail. When the predetermined number of times is one, a single signal is read from the detection pixel 104. Thus, the signal processing unit 170 sets a value of the signal as the correction value Od. When the predetermined number of times is more than one, the signal processing unit 170 sets an average value of the plurality of read signals as the correction value Od. Instead of the average value, other statistic values may be used. The correction value Oc is also similarly decided based on the signal read from the correction pixel 107. The signal processing unit 170 stores the thus decided correction values Od and Oc in the storage unit 172 which can be used for subsequent processing.

When the read operation is ended once or more, at time t5, the control circuit 180 transmits the start possible signal to the radiation source interface 502. The decision on the above-described correction values Od and Oc may be performed before the transmission of the start possible signal or may be performed after the transmission. After the start possible signal is transmitted, the control circuit 180 repeatedly executes the above-described read operation. The signal processing unit 170 measures an irradiation amount DOSE of the radiation for each read operation and determines whether or not the integrated value exceeds a threshold. After time t5, the radiation irradiation is started from time t6.

A method of deciding the irradiation amount DOSE will be described below. A value of the signal read by the latest read operation from the detection pixel 104 is denoted as Sd. A value of the signal read by the latest read operation from the correction pixel 107 is denoted as Sc. The signal processing unit 170 calculates DOSE by applying Sd, Sc, Od, and Oc to the following expression (1).

DOSE=(Sd−Od)−(Sc−Oc) (1)

In this expression, DOSE is decided based on a difference between the value Sc of the signal read from the correction pixel 107 after the start possible signal is transmitted and the correction value Oc decided based on the signal read from the correction pixel 107 before the start possible signal is transmitted.

In addition, the signal processing unit 170 may calculate DOSE by applying Sd, Sc, Od, and Oc to the following expression (2) instead of the expression (1).

DOSE=Sd−Od×Sc/Oc (2)

In the following expression, DOSE is decided based on a ratio between the value Sc of the signal read from the correction pixel 107 after the start possible signal is transmitted and the correction value Oc decided based on the signal read from the correction pixel 107 before the start possible signal is transmitted.

As illustrated in FIG. 6, the signal read from the detection pixel 104 largely changes over time immediately after the end of the reset operation (immediately after time t3) and is to be stabilized over an elapse of time (for example, in approximately 100 ms). For this reason, even when DOSE is calculated by using only Sd and Od obtained from the detection pixel 104, it is not possible to sufficiently remove an offset amount. When start of the read operation for obtaining the correction value Od is delayed until the signal read from the detection pixel 104 is stabilized, a time period from the transmission of the start request signal to the actual radiation irradiation start (time period from time t2 to time t6, that is a so-called exposure delay) is lengthened.

According to the present embodiment, the irradiation amount DOSE is decided by further using the values (Sc and Oc) of the signals read from the correction pixel 107. Since the correction pixel 107 has a very low sensitivity to radiation, the value Sc of the signal read from the correction pixel 107 after the radiation irradiation start can be regarded as an offset component of the value Sd of the signal read from the detection pixel 104.

Furthermore, according to the present embodiment, the irradiation amount DOSE is decided based on the correction values Od and Oc based on the signals read from the detection pixel 104 and the correction pixel 107 before the radiation irradiation start. With this configuration, it is possible to correct inherent characteristic differences of the respective pixels (differences in detection circuit channels, differences in parasitic resistances, and parasitic capacitances of the respective pixels, and the like).

When the accumulated irradiation amount has reached

the threshold at time t7, the control circuit 180 transmits the end request signal to the radiation source interface 502. Instead of this, the control circuit 180 may estimate a point in time when the accumulated irradiation amount is to reach the threshold and may transmit the end request signal at this estimated time. At time t8, the radiation source interface 502 causes the radiation source 501 to end the radiation irradiation in response to reception of the end request signal.

In the above-described example, the control circuit 180 starts the read operation to be performed a predetermined number of times for deciding the correction values Od and Oc immediately after the end of the reset operation. Instead of this, after the end of the reset operation, the control circuit 180 may start the read operation to be performed a predetermined number of times since an elapse of a predetermined time period (for example, several ms to several tens of ms). With this configuration, it is possible to suppress the read of the signal during a period in which the signal value particularly largely fluctuates due to switching of operations.

When the radiation irradiation ends at time t8, the control circuit 180 performs the reset operation of the plurality of pixels up to time t9 similarly as in time t0.

When the reset operation is ended at time t9, the control circuit 180 performs the read operation of the detection pixel 104 and the correction pixel 107 a predetermined number of time that is at least once such that the accumulation period becomes the same in t3 to t8 and t9 to t10. The signal processing unit 170 decides the correction value Od based on the signal read from the detection pixel 104 by the read operation performed a predetermined number of times and the correction value Oc based on the signal read from the correction pixel 107 by this read operation performed a predetermined number of times.

In the explanation so far, the example has been described in which shooting is performed once. Hereinafter, a case will be described where shooting at t10 and after the subsequent times is performed. In a case where there is subsequent shooting in which the radiation irradiation is ended based on the reach of the threshold of the accumulated irradiation amount performed at time t7, by obtaining the above-described correction values Od and Oc during the shooting, it is possible to perform the highly precise AEC control while an impact on the shooting timing is reduced. Such shooting is performed in a case, for example, where shooting is performed a plurality of times in succession for group medical examination or the like.

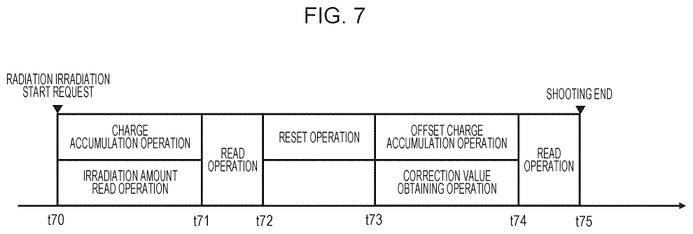

FIG. 7 is an explanatory diagram for describing an operation of obtaining the correction value during shooting. In FIG. 7, the read operation for deciding the correction values Od and Oc is performed in parallel with the processing operation of obtaining an offset output from the image capture pixel 101 in t72 to t74 from the start of the radiation irradiation to the end of the shooting.

In t70 to t71, the control circuit 180 performs control such that the charges of the image capture pixel 101 are accumulated and also performs the read operation of the irradiation amount from the detection pixel 104 and the correction pixel 107.

In t71 to t72, the control circuit 180 performs control such that the charges based on the radiation which are accumulated in the image capture pixel 101 are read as an image. In t72 to t73, the control circuit 180 performs the above-described reset operation as the operation in t8 to t9 of FIG. 6. In t73 to t74, the control circuit 180 performs the above-described read operation of the offset signal as the operation in t9 to t10 of FIG. 6. In t74 to t75, the control circuit 180 performs the read operation of the charges for a purpose of performing the offset correction of the image with regard to the charges of the image capture pixel 101 which are accumulated in t73 to t74.

Herein, the read operation for deciding the correction values Od and Oc may be implemented at any timing in a period from t72 to t74. By obtaining the correction value in parallel with the processing operation performed in this period, a frequency of obtaining the correction value before the radiation irradiation is started is lowered, so that an advantage is attained that the exposure delay may be reduced.

In addition, when an ROI is designated, with regard to targets in which the correction value is to be obtained, only pixels where the read has been performed from the detection pixel 104 and the correction pixel 107 which are set as the read targets in t70 to t71 may be set as targets. With this setting, since the correction values can be obtained without reading an unnecessary correction value, an extension of a cycle time up to the subsequent shooting may be reduced.

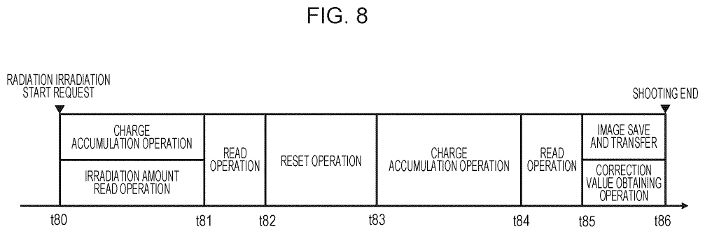

FIG. 8 is an explanatory diagram for describing an operation of obtaining the correction value in parallel with a processing operation after the shooting such as image transfer.

In t80 to t83, the control circuit 180 performs

processing similar to that in t70 to t73 of FIG. 7. In t83 to t84, the control circuit 180 performs control such that charges are accumulated in the image capture pixel 101. In t84 to t85, the control circuit 180 performs processing similar to that in t74 to t75 of FIG. 7.

In t85 to t86, the control circuit 180 stores a shot image based on the radiation read in t81 to t82 and an offset image read in t84 to t85 in the storage unit 172. Thereafter, the images are transferred to the controller 504 via the communication interface 503. Herein, an image to be saved or transmitted may be an image obtained by performing image processing such as offset correction on the shot image based on the radiation.

In addition, the control circuit 180 performs an operation of saving the above-described image in t85 to t86 and also performs the read operation for deciding the correction values Od and Oc from the detection pixel 104 and the correction pixel 107.

As described above, when the correction value is to be obtained in parallel with post-processing during the shooting, an advantage is attained that the exposure delay may be reduced similarly as in FIG. 7.

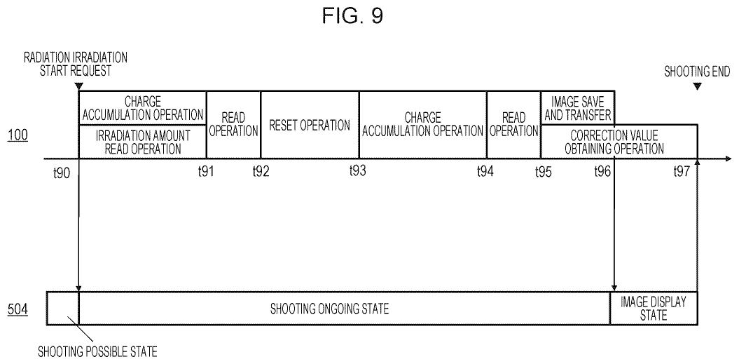

FIG. 9 is a diagram for describing a flow of obtaining the correction value in parallel with a processing operation of displaying an image to the controller 504 during the shooting. At t90, the control circuit 180 receives the radiation irradiation start request from the radiation source interface 502 and transmits information indicating that the shooting has been started to the controller 504. The controller 504 is put into a shooting ongoing state corresponding to a state in which the shooting has been started.

In t90 to t96, the control circuit 180 performs processing similar to that of FIG. 8. It is noted however that at t96, the controller 504 is put into a state in which the image can be displayed when all the image data is received from the radiation image capture apparatus 100 or information indicating that all the image save has been completed is received. The control circuit 180 continues the read operation for deciding the correction values Od and Oc from the detection pixel 104 and the correction pixel 107.

At t97, information indicating that the controller 504 is no longer capable of displaying the image due to preparation or the like for performing the subsequent shooting is transmitted to the radiation image capture apparatus 100. When the control circuit 180 receives the information indicating that the controller 504 is no longer capable of displaying the image, the control circuit 180 stops the read operation for deciding the correction values Od and Oc from the detection pixel 104 and the correction pixel 107.

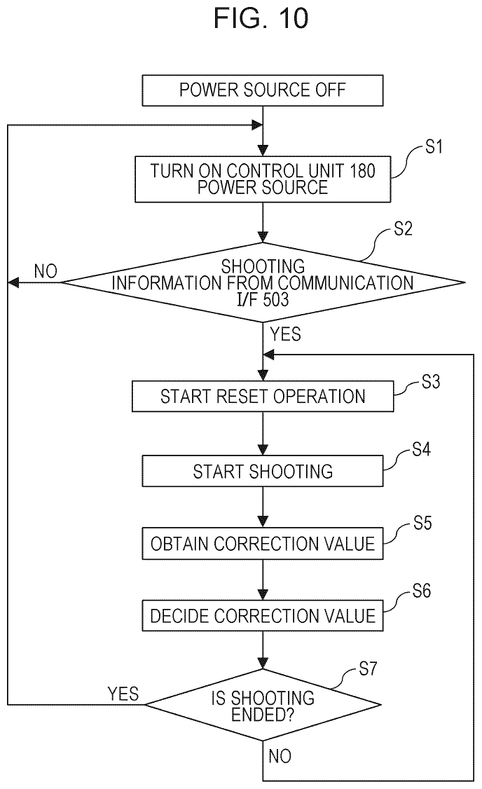

FIG. 10 is a flow of an operation of obtaining a correction value during the shooting.

In step S1, first, the radiation image capture apparatus 100 turns ON a power source of the control circuit 180 to shift to a state in which communication with the radiation generation apparatus can be performed.

In step S2, in a case where the shooting information is transmitted to the radiation image capture apparatus 100 from the radiation generation apparatus via the communication interface 503, the reset operation in step S3 is started for preparing the shooting to perform an operation of initializing the image capture region IR. In a case where the shooting information is not transmitted, only the control circuit 180 in step S1 keeps a state in which the power source is ON to stand by until the shooting information is transmitted while power consumption is reduced.

In step S3, power is supplied to the read circuit and the drive circuit to start the reset operation for removing charges accumulated in each of the pixels. This reset operation is repeatedly performed until the start request is received. When the start request is received, the shooting of the radiation image in step S4 is started. In step S5 corresponding to a period after the image based on the radiation is obtained until the shooting is ended, an offset output is obtained during the shooting. As a method of obtaining the offset output, a method of performing the read operation is adopted to directly obtain the offset output, or the offset output may be indirectly obtained through an estimation from an output in correlation with the offset output.

In step S6, the correction value for performing the radiation irradiation stop determination of the subsequent shooting is decided.

In step S7, when there is subsequent shooting in succession, the flow returns to S3, and there is no subsequent shooting, the flow returns to S1. It is noted that timing indicated by a phrase “during the shooting” in the explanation of the present embodiment refers to steps S4 to S7 in the flow of FIG. 10, that is, from a point in time when the control circuit 180 has received the start request signal until each processing related to the shooting performed by the control circuit 180 is ended.

Immediately after the shot image based on the radiation is obtained, while the charges may remain in the detection pixel 104, a time period such as an accumulation time period for obtaining an offset image may be short. Thus, the correction value may be decided through an estimation. For example, by using a least square method or a data imputation technique from results of the offset output performed twice or more, it is possible to decide the correction value in which residual charge components have been reduced.

As described above, according to the present embodiment, by obtaining the correction value during the shooting, the frequency of obtaining the correction value is set to be a requisite minimum, and while the precision of the AEC is kept, it is possible to reduce the impact on the shooting timing.

### Second Embodiment

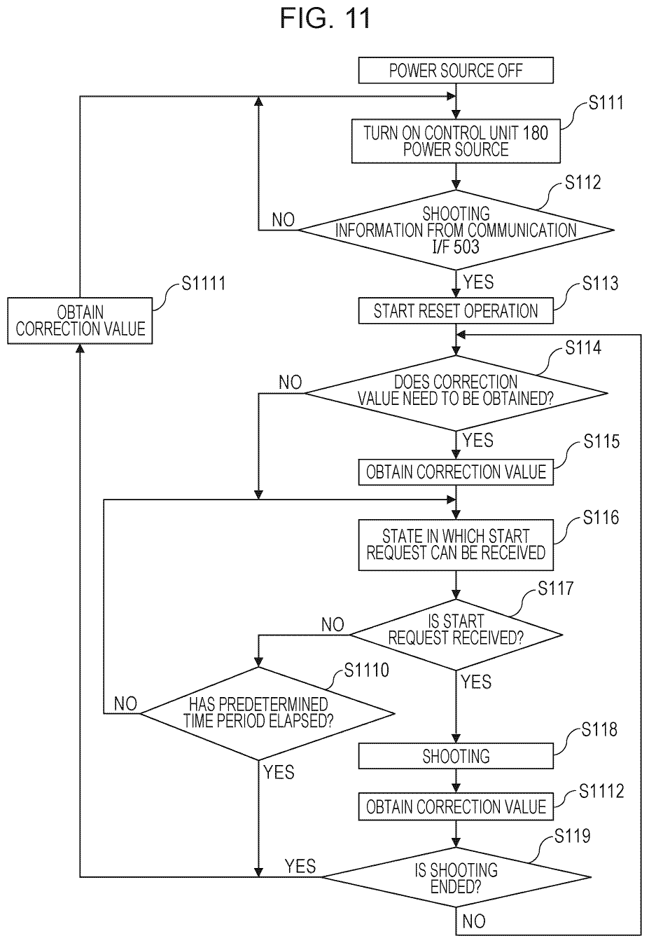

FIG. 11 is a flow for describing an operation of the radiation image capture apparatus according to a second embodiment. According to the present embodiment, in addition to the first embodiment, the correction value is obtained before the start of the shooting.

Steps S111 to S113 are similar to steps S1 to S3 of FIG. 10. According to the present embodiment, it is determined whether or not the correction value in step S114 needs to be obtained during the reset operation.

A criterion for the determination is whether or not a parameter impacting the correction value is in a desired range as compared with the last time when the correction value is obtained. The parameter impacting the correction value includes an elapsed time period since the correction value is obtained last time, a temperature of a sensor, or the like. In addition, the outputs of the detection pixel 104 and the correction pixel 107 may be obtained similarly as in the obtainment of the correction value, and the determination may be performed based on a fluctuation amount from the correction value obtained last time. As a method of obtaining the outputs of the detection pixel 104 and the correction pixel 107, a method of performing the read operation is adopted to directly obtain the outputs, or the outputs may be indirectly obtained through an estimation from an output in correlation with the offset output.

As a result of the determination, in a case where the correction value does not need to be obtained, the reset operation is continued, and after the image capture region IR is stabilized, the state is shifted to a state in which the start request can be received in step S116. Until the start request is received in step S117, the reset operation is repeated.

In a case where the correction value needs to be obtained, to obtain the correction value in step S115, the shift from the reset operation to the operation of deciding the correction value is performed to obtain the correction value. After the obtainment of the correction value is completed, the operation is switched to the reset operation again to shift to a state in which the start request can be received in step S116. Until the start request is received in step S117, the reset operation is repeated.

According to the present embodiment, the example has been illustrated in which the determination is performed on whether or not the correction value is obtained after the start of the reset operation, but the determination on whether the correction value needs to be obtained may be performed at a point in time when the shooting information from the radiation generation apparatus is received. In this case, after the determination is performed, the operation is shifted to the reset operation, and when necessary, the correction value is obtained.

When the start request is received in step S117, shooting of the radiation image in step S118 is performed. After the shooting, in a case where subsequent shooting information is received, the determination on whether or not the correction value needs to be obtained in step S114 is performed again, and when necessary, after the correction value is obtained, the state is shifted to a state in which the start request can be received to perform the shooting.

After the shift to the state in which the start request can be received in step S116, in a case where the shooting information is not transmitted in step S119 or a case where a predetermined time period has elapsed in step S1110, after the correction value is obtained, the state is shifted to a state in which only the power source of the control circuit 180 is ON in step S1. With such a configuration, it is possible to stand by while the power consumption of the radiation image capture apparatus 100 is reduced.

In step S118, the shooting is started. Similarly as in step S6 of FIG. 10, the correction value for performing the radiation irradiation stop determination on the subsequent shooting is decided in step S1112. In a case where the shooting is ended in step S119, the correction value is obtained in step S1111 through the processing similar to that in S1112. In a case where the shooting is not ended, the processing similar to that in step S4 is performed again. Herein, an accumulation, an average, or the like of the correction values obtained in step S1111 and step S1112 may be taken.

With such a configuration, the correction value can be obtained without an impact on the shooting timing. In addition, by performing the determination in step S114, since it is possible to lower the frequency that the correction value needs to be obtained, the impact on the shooting timing can be reduced while the precision of the AEC is kept.

### Third Embodiment

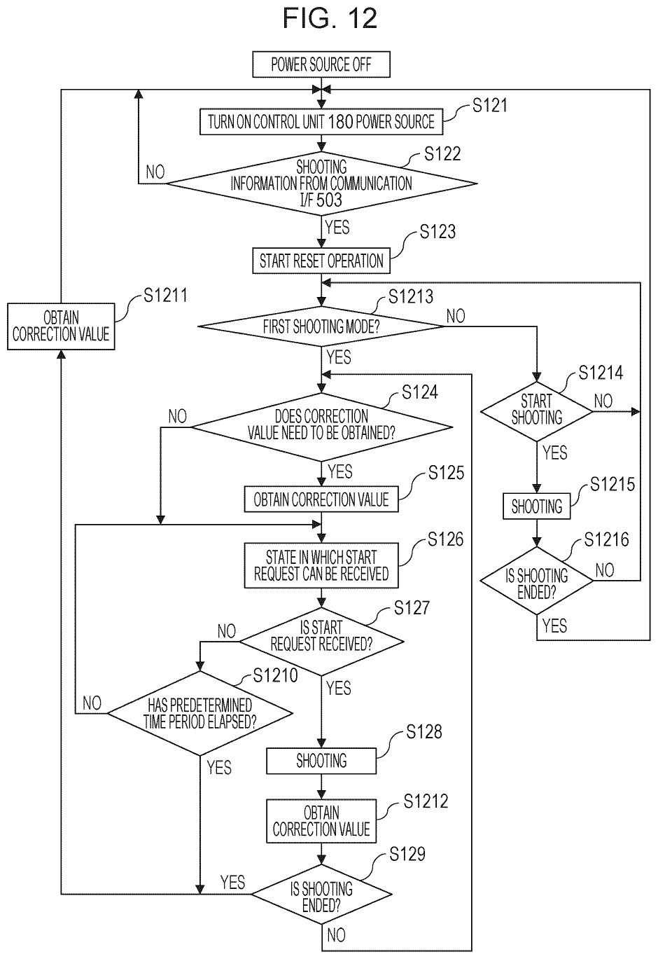

FIG. 12 is a flow for describing an operation of the radiation image capture apparatus according to a third embodiment. According to the present embodiment, in addition to the second embodiment, in the radiation image capture apparatus 100 which can perform shooting in a plurality of shooting modes, the correction value is obtained in only a particular operation mode.

According to the present embodiment, as described in the first embodiment, the radiation image capture apparatus 100 has a plurality of shooting modes. For example, the radiation image capture apparatus 100 has the above-described first to third shooting modes, the exposure decision unit does not obtain at least one of the first correction value and the second correction value is not obtained in a mode excluding the first shooting mode.

In steps S121 to S1212, the processing similar to that in steps S111 to S1112 of FIG. 11 according to the second embodiment is performed.

In S1213, in a case where the mode is the first shooting mode, the control circuit 180 performs the processing in S124, and in a case where the mode is not the first shooting mode, for example, a case where the mode is the second shooting mode, the control circuit 180 determines in S1214 whether the shooting is to be started depending on the presence or absence of the radiation irradiation or the like. In a case where the shooting is not started, the processing in S1213 is performed. In a case where it is determined in S1214 that the shooting is to be started, the shooting is started in S1215. In a case where the shooting is ended in S1216, the processing in S1 is implemented, and in a case where the shooting is not ended, the processing in S1213 is performed.

Since the correction value is not obtained in a mode excluding the first shooting mode, the frequency that the correction value needs to be obtained can be lowered, so that the impact on the shooting timing can be reduced.

### Fourth Embodiment

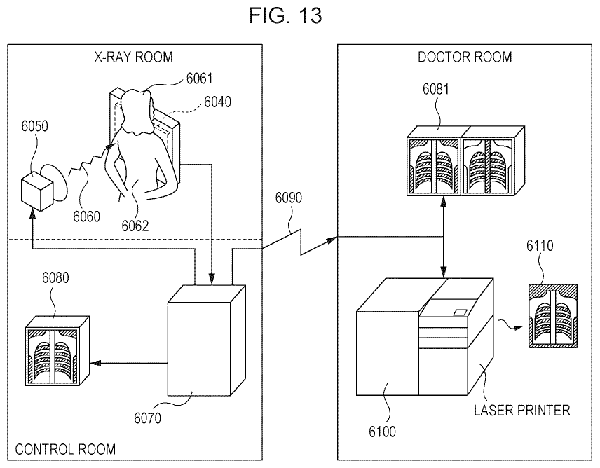

Hereinafter, an example in which the radiation image capture apparatus 100 is applied to a radiation image shooting system will be described with reference to FIG. 13. An X-ray 6060 generated in an X-ray tube 6050 serving as a radiation source transmits through a chest region 6062 of a patient or an examinee 6061 to be incident on a radiation image capture apparatus 6040 represented by the radiation image capture apparatus 100 described above. This incident X-ray includes internal body information of the examinee 6061.

The scintillator emits light in response to the incidence of the X-ray, and the light is subjected to photoelectric conversion by the photoelectric conversion element to obtain electric information. This information can be digitally converted to be subjected to image processing by an image processor 6070 serving as a signal processing unit and observed on a display 6080 serving as a display unit in a control room.

In addition, this information can be transferred to a remote place by a transmission processing unit such as a telephone line 6090. This information can be displayed on a display 6081 serving as a display unit in a doctor room or the like in another place or saved in a recording unit such as an optical disk and also diagnosed by a doctor in a remote place. In addition, this information can be recorded on a film 6110 serving as a recording medium by a film processor 6100 serving as a recording unit.

### Other Embodiments

The present disclosure can also be realized by processing in which a program for realizing the above-described functions is supplied to a system or an apparatus via a network or a storage medium, and one or more processors in a computer of the system or the apparatus reads and executes a program.

In addition, various recording media such as a flexible disk, an optical disk (for example, a CD-ROM and a DVD-ROM), an opto-magnetic disk, a magnetic tape, a nonvolatile memory (for example, a USB memory), and a ROM can be used as the recording medium. In addition, a program for implementing the above-described functions may be downloaded via a network to be executed by the computer.

In addition, the present disclosure is not limited to a case where the functions of the above-described embodiments are realized by executing a program code read by the computer. The present disclosure also includes a case where an operating system (OS) or the like running on the computer performs part or all of actual processing based on instructions of the program code, and the functions of the above-described embodiments are realized by the processing.

Furthermore, the program code read from the recording medium may be written to a memory provided in a function expansion board inserted into the computer or a function expansion unit connected to the computer. The present disclosure also includes a case where a central processing unit (CPU) or the like provided in the function expansion board or the function expansion unit performs part or all of actual processing based on instructions of the program code, and the above-described functions are realized by the processing.

According to at least one of the embodiments of the present disclosure, the offset component to be used when the radiation dose during the radiation irradiation is monitored can be precisely obtained while the delays of the start timing and the end timing of the shooting are reduced.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

1. A radiation image capture apparatus comprising:
an image capture unit including a plurality of pixels which include an image capture pixel configured to obtain a radiation image and a detection pixel configured to detect a radiation dose;
means configured to execute stop processing for stopping radiation irradiation by a radiation irradiation apparatus based on an output of the detection pixel; and
means configured to execute, in first image capture including a front-end process of causing radiation to be incident on the image capture unit and a post process for obtaining a radiation image after end of the radiation irradiation, obtaining processing for obtaining predetermined correction information based on an output of the detection pixel in parallel with predetermined processing for obtaining the radiation image which is executed in the post process, wherein
the predetermined correction information is used in the stop processing in second image capture which is to be performed after the first image capture.

2. The radiation image capture apparatus according to claim 1, wherein the predetermined processing is processing to be performed after a read operation of reading an output from the image capture pixel is ended.

3. The radiation image capture apparatus according to claim 2, wherein the predetermined processing is processing of obtaining an offset image for correcting an image obtained in the first image capture.

4. The radiation image capture apparatus according to claim 2, wherein the predetermined processing is processing of transferring an image obtained in the first image capture from the radiation image capture apparatus to the outside.

5. The radiation image capture apparatus according to claim 2, wherein the predetermined processing is processing of standing by until an image obtained in the first image capture is transferred from the radiation image capture apparatus to an external device to be displayed on the external device.

6. The radiation image capture apparatus according to claim 1, wherein the predetermined correction information is information in which an impact from charges remaining in the detection pixel is reduced after the read operation is performed.

7. The radiation image capture apparatus according to claim 6, wherein the predetermined correction information is information based on a plurality of offset outputs obtained from the detection pixel.

8. The radiation image capture apparatus according to claim 1, wherein it is determined whether or not the predetermined correction information is to be obtained based on at least one of an elapsed time period since obtainment of the predetermined correction information which has been obtained before the first image capture, a temperature of the radiation image capture apparatus, and a fluctuation of an output of the pixel while the radiation irradiation is not performed after the predetermined correction information is obtained before the first image capture.

9. The radiation image capture apparatus according to claim 1, wherein the predetermined correction information is information obtained based on an output of the detection pixel during a period in which radiation irradiation is not performed before the first image capture and information obtained based on an output of the detection pixel during a period of the post process in the first image capture.

10. The radiation image capture apparatus according to claim 1, wherein
the radiation image capture apparatus has a first image capture mode in which the stop processing is performed and a second image capture mode in which the stop processing is not performed, and
the radiation image capture apparatus does not perform the obtaining processing in the second image capture mode.

11. The radiation image capture apparatus according to claim 1, wherein the detection pixel includes a plurality of detection pixels in the plurality of pixels, and in the obtaining processing, the predetermined correction information is obtained based on an output of a detection pixel in a region of interest among the plurality of detection pixels.

12. The radiation image capture apparatus according to claim 1, wherein
the plurality of pixels include a correction pixel configured to have a lower sensitivity to radiation than that of the detection pixel, and
the radiation image capture apparatus further comprises means configured to execute further obtaining processing for obtaining further correction information based on an output of the correction pixel.

13. The radiation image capture apparatus according to claim 12, wherein the further obtaining processing is performed in parallel with the predetermined processing.

14. The radiation image capture apparatus according to claim 12, further comprising:
means configured to determine whether or not the predetermined correction information and the further correction information are obtained based on at least any one of an elapsed time since obtainment of the predetermined correction information and the further correction information which have been obtained before the first image capture, a temperature of the radiation image capture apparatus, and fluctuations of outputs of the detection pixel and the correction pixel while radiation irradiation is not performed since the obtainment of the predetermined correction information and the further correction information before the first image capture.

15. The radiation image capture apparatus according to claim 12, wherein the predetermined correction information and the further correction information are obtained from information obtained based on an output of the detection pixel and an output of the correction pixel during a period in which radiation irradiation is not performed before the first image capture and information obtained based on an output of the detection pixel and an output of the correction pixel during a period in which radiation irradiation is not performed in the first image capture.

16. The radiation image capture apparatus according to claim 12, wherein
the radiation image capture apparatus has a first image capture mode in which the stop processing is performed and a second image capture mode different from the first image capture mode, and
the radiation image capture apparatus does not obtain at least one of the predetermined correction information and the further correction information in the second image capture mode.

17. The radiation image capture apparatus according to claim 12, wherein
the detection pixel includes a plurality of detection pixels and the correction pixel includes a plurality of correction pixels in the plurality of pixels, and
in the obtaining processing, the predetermined correction information is obtained based on a detection pixel in a region of interest among the plurality of detection pixels, and in the further obtaining processing, the further correction information is obtained based on a correction pixel in the region of interest among the plurality of correction pixels.

18. A radiation image capture system comprising:
the radiation image capture apparatus according to claim 1; and
a signal processing unit configured to process a signal from the radiation image capture apparatus.

19. A control method for a radiation image capture apparatus provided with an image capture unit including a plurality of pixels which include an image capture pixel configured to obtain a radiation image and a detection pixel configured to detect a radiation dose and configured to execute stop processing for stopping radiation irradiation by a radiation irradiation apparatus based on an output of the detection pixel, the control method comprising:
executing, in first image capture including a front-end process of causing radiation to be incident on the image capture unit and a post process for obtaining a radiation image after end of the radiation irradiation, obtaining processing for obtaining predetermined correction information in parallel with predetermined processing for obtaining the radiation image which is executed in the post process, wherein
the predetermined correction information is used in the stop processing in second image capture which is to be performed after the first image capture.

20. A non-transitory computer-readable medium for causing a computer to execute a control method for a radiation image capture apparatus provided with an image capture unit including a plurality of pixels which include an image capture pixel configured to obtain a radiation image and a detection pixel configured to detect a radiation dose and configured to execute stop processing for stopping radiation irradiation by a radiation irradiation apparatus based on an output of the detection pixel, the control method comprising:
executing, in first image capture including a front-end process of causing radiation to be incident on the image capture unit and a post process for obtaining a radiation image after end of the radiation irradiation, obtaining processing for obtaining predetermined correction information in parallel with predetermined processing for obtaining the radiation image which is executed in the post process, wherein
the predetermined correction information is used in the stop processing in second image capture which is to be performed after the first image capture.
